(19) United States
(12) Patent Application Publication
(10) Pub. No.: US 2022/0022731 A1
(43) Pub. Date: Jan. 27, 2022

(54) TUBE FOR MEDICAL DEVICE, AND MEDICAL DEVICE

(71) Applicant: OLYMPUS CORPORATION, Tokyo (JP)

(72) Inventor: Kiyoshi TAKAO, Hachioji-shi (JP)

(73) Assignee: OLYMPUS CORPORATION, Tokyo (JP)

(21) Appl. No.: 17/492,713

(22) Filed: Oct. 4, 2021

Related U.S. Application Data
(63) Continuation of application No. PCT/JP2020/014449, filed on Mar. 30, 2020.

(30) Foreign Application Priority Data
Apr. 9, 2019 (JP) 2019-074162

(51) Int. Cl. A61B 1/005; A61B 1/00

(52) U.S. Cl. CPC A61B 1/0051; A61B 1/0011

(57) ABSTRACT

A tube for a medical device includes an inner layer tube formed from an elastomer or a resin having flexibility and having a non-parallel groove with respect to an axial direction formed on an outer circumferential surface of the inner layer tube; an outer layer formed from an elastomer resin softer than a base material of the inner layer tube and configured to fill the groove while covering an outer circumference of the inner layer tube; and a braid formed from metal wires and only arranged in locations other than the groove in the outer layer, wherein the outer layer is divided to at least two layers, the innermost layer is a layer filling the groove of the inner layer tube, and the braid is arranged only in the layers other than the innermost layer.

### TECHNICAL FIELD

The present disclosure relates to a tube for medical device and a medical device.

The present application is a continuation application of PCT International Application No. PCT/JP2020/014449, filed on Mar. 30, 2020, whose priority is claimed on Japanese Patent Application No. 2019-074162, filed on Apr. 9, 2019. The contents of the PCT International Application and the Japanese Patent Application are incorporated herein by reference.

### BACKGROUND ART

In recent years, there is a strong demand for the improvement of the characteristic such as flexibility, kink resistance, and wear resistance in a tube for a medical device.

For example, in a case of a tube for a medical device used as a channel tube for an endoscope, it is required to improve the flexibility and the kink resistance for the purpose of realizing excellent operability. Further, it is required to improve the wear resistance for the purpose of preventing wear due to the repeated insertion and removal of the treatment tool such as forceps.

For example, the treatment tool insertion channel disclosed in Japanese Unexamined Patent Application, First Publication No. H3-205022, a net made of stainless steel wire is attached to a tube main body made of the urethane resin having an inner surface coating layer made of the Teflon (registered trademark) formed on the inner surface thereof. A coating layer made of the urethane resin is formed on the attachment portion of the net. Since the net expands and contracts easily when being bent, the resistance to bending is small. Further, the net has the shape retainability.

The tube for an endoscope disclosed in Japanese Unexamined Patent Application, First Publication No. 2010-29435 is configured to include a tube main body made from the fluororesin, a reinforcing tape wound around and fixed to the outer circumferential surface of the tube main body, and an outer skin made from the polyurethane and covering the tube main body from above the reinforcing tape. The reinforcing tape includes a reinforcing net formed from the polyester resin wires such that the anisotropy is imparted to the rigidity in the axial direction and the circumferential direction.

### SUMMARY

According to a first aspect of the present disclosure, a tube for a medical device includes an inner layer tube formed from an elastomer or a resin having flexibility and having a non-parallel groove with respect to an axial direction formed on an outer circumferential surface of the inner layer tube; an outer layer formed from an elastomer resin softer than a base material of the inner layer tube and configured to fill the groove while covering an outer circumference of the inner layer tube; and a braid formed from metal wires and only arranged in locations other than the groove in the outer layer. The outer layer is divided to at least two layers, wherein the innermost layer is a layer filling the groove of the inner layer tube, and the braid is arranged only in the layers other than the innermost layer.

According to a second aspect of the present disclosure, in the tube for a medical device according to the first aspect, a material of the inner tube may include polytetrafluoroethylene (PTFE).

According to a third aspect of the present disclosure, in the tube for a medical device according to the first aspect, a material of the outer layer may include fluororubber.

According to a fourth aspect of the present disclosure, a medical device includes the tube for a medical device according to the first aspect.

According to a fifth aspect of the present disclosure, in the medical device according to the fourth aspect, the medical device may be an endoscope.

### BRIEF DESCRIPTION OF DRAWINGS

FIG. 1 is a perspective view schematically showing a configuration example of a medical device according to a first embodiment of the present disclosure.

FIG. 2 is a partial cross-sectional view schematically showing a configuration example of a tube for a medical device according to the first embodiment of the preset disclosure.

FIG. 3 is a view showing the effects of the tube for a medical device.

FIG. 4 is a schematic view showing the effects of a tube for a medical device according to a comparison example.

FIG. 5 is a partial cross-sectional view schematically showing a configuration example of a tube for a medical device according to a second embodiment of the preset disclosure.

FIG. 6 is a partial cross-sectional view schematically showing a configuration example of a tube for a medical device according to a modification (first modification) of the present embodiment.

FIG. 7 is a partial cross-sectional view schematically showing a configuration example of a tube for a medical device according to a first comparison example.

FIG. 8 is a schematic view showing an experiment method of the wear resistance evaluation.

FIG. 9 is a schematic view showing an experiment method of the flexibility evaluation.

### DESCRIPTION OF EMBODIMENTS

Hereinafter, each embodiment of the present disclosure will be described with reference to the accompanying drawings. In all the drawings, even if the embodiments are different, the same or corresponding members are designated by the same reference numerals, and common description will be omitted.

### First Embodiment

Hereinafter, a tube for a medical device and a medical device according to an embodiment of the present disclosure will be described.

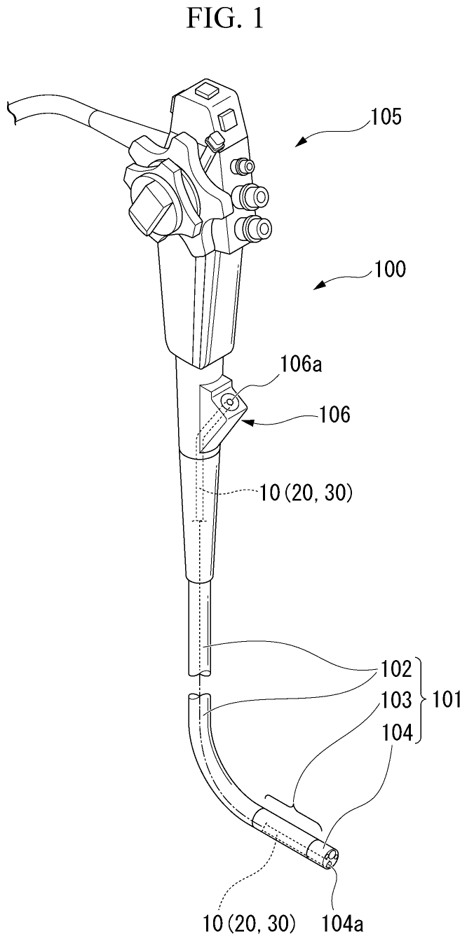

FIG. 1 is a schematic perspective view showing a configuration example of a medical device according to a first embodiment of the present disclosure.

As shown in FIG. 1, an endoscope 100 (medical device) according to the present embodiment includes an insertion portion 101 and an operation portion 105.

The insertion portion 101 is configured to be inserted into a patient's body. The insertion portion 101 is tubular. The insertion portion 101 has flexibility. The insertion portion 101 has a distal end portion 104, a bending portion 103, and a flexible tube portion 102 in such a sequence from a distal end side in the insertion direction. Inside the insertion portion 101, a channel tube 10 (medical device tube) for inserting through a treatment tool is provided in a longitudinal direction.

The distal end portion 104 is arranged at the most distal end portion of the endoscope 100. The distal end portion 104 has a columnar outer shape. The distal end portion 104 includes an image sensor element and an image optical system inside. An imaging window and an illumination window are provided at the distal end of the distal end portion 104.

Further, an opening 104a that communicates with the inside of the channel tube 10 is formed at the distal end of the distal end portion 104.

The bending portion 103 is connected to the proximal end side of the distal end portion 104. The bending portion 103 is configured to change the orientation of the distal end portion 104. The bending portion 103 is a tubular portion and the bending portion 103 is bendable.

The bending portion 103 includes, for example, a plurality of joint rings. Each of the joint rings is annular shaped. Each joint ring is rotatably connected to the adjacent joint ring. In the bending portion 103, a plurality of angle wires are inserted inside the plurality of joint rings.

Further, members such as electric wirings, a light guide, and a channel tube 10 are accommodated inside the bending portion 103. The electrical wirings ere connected to the image sensor at the distal end portion 104. The light, guide extends close to the illumination window.

The channel tube 10 is an elongated tubular member that configures the treatment tool channel through which a treatment tool (not shown) is inserted. The distal end of the channel tube 10 is connected to the opening 104a. The detailed configuration of the channel tube 10 will be described later.

The electrical wirings, the light guide, and the channel tube 10 are inserted into the flexible tube portion 102 described later and extend to the operation portion 105 described later.

The flexible tube portion 102 is a tubular portion configured to connect the bending portion 103 and the operation portion 105.

The flexible tube portion 102 includes, for example, a serpentine tube and an outer skin (not shown). The serpentine tube is a member formed by spirally wounding a metal or resin strip-shaped member. The outer skin is arranged on the outermost side of the flexible tube portion 102. The outer skin is a tube that covers the outer circumference of the serpentine tube. The outer skin is made from a flexible resin material.

Although not particularly shown in figures, two systems of angle wires including at least a first angle wire and a second angle wire are arranged inside the flexible tube portion 102. Each angle wire is inserted through a coil sheath. Each angle wire extends from the bending portion 103 toward the proximal end side.

Similar to the bending portion 103, members such as the above-described electrical wirings, light guide, and channel tube 10 are inserted into the flexible tube portion 102.

The operation portion 105 is a device portion configured for an operator to operate the endoscope 100. Examples of the operations performed by the operator through the operation portion 105 include an operation of pulling the angle wire for the purpose of changing the bending amount of the bending unit 103. The operation portion 105 includes an operation portion main body grasped by the operator and various operation members provided on the operation portion main body. For example, various operation members may be operation knobs, operation switches, and the like.

A treatment tool insertion portion 106 is provided at the distal end of the operation portion 105.

The treatment tool insertion portion 106 has an opening 106a into which the treatment tool is inserted. The proximal end of the channel tube 10 is connected to the insertion port 106a.

Next, the detailed configuration of the channel tube 10 according to the present embodiment will be described.

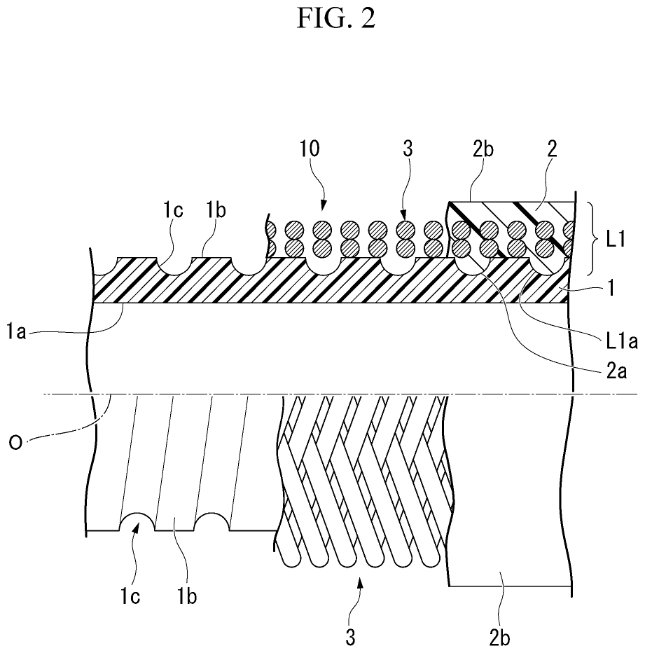

FIG. 2 is a schematic partial cross-sectional view showing a configuration example of a medical device tube according to the first embodiment of the present disclosure.

As shown in FIG. 2, the channel tube 10 according to the present embodiment includes an inner layer tube 1 and an outer layer portion L1 (outer layer).

The channel tube 10 is a flexible tube for a medical device. The channel tube 10 according to the present embodiment is used in the endoscope 100 as a treatment tool channel through which, for example, a treatment tool or the like is inserted.

However, the medical device in which the channel tube 10 is used is not limited to the endoscope. The channel tube 10 is particularly suitable for applications to insert a rigid member therein. For example, the channel tube 10 may be used for an air-water supply tube, a catheter for a treatment tool, or the like.

The inner layer tube 1 is a resin tubular member having a through hole formed inside the inner layer tube 1 and extending in the longitudinal direction. The length of the inner layer tube 1 is not particularly limited as long as the inner layer tube 1 can be inserted into the patient's body through the endoscope 100. For example, the length of the inner layer tube 1 may be equal to or more than 400 mm and equal to or less than 3000 mm.

At the inner side of an inner circumferential surface 1a forming the through hole, for example, a shaft-shaped or tubular insertion member such as a treatment tool or a catheter and the like may be inserted.

The inner circumferential surface 1a is repeatedly washed. Taking the ease of cleaning into consideration, the inner circumferential surface 1a is more preferably to be a smooth surface. When the inner circumferential surface 1a is a smooth surface, it is possible to insert the treatment tool or the like into the inner circumferential surface 1a more smoothly.

For the purpose of making the inner circumferential surface 1a to be a smooth surface, at least the portion exposed as the inner circumferential surface 1a may be made of a non-porous material.

In the example shown in FIG. 2, the inner circumferential surface 1a is a smooth cylindrical surface.

An outer circumferential portion of the inner layer tube 1 is formed by an outer circumferential surface 1b. The outer circumferential surface 1b forms the outermost side portion of the inner layer tube 1.

A thickness of the inner layer tube 1 may be equal to or more than 0.1 mm and equal to or less than 1.0 mm. The thickness of the inner layer tube 1 is more preferably to be equal to or more than 0.3 mm and equal to or less than 0.5 mm.

A groove 1c recessed toward the inner circumferential surface 1a is formed on the outer circumferential surface 1b. The groove 1c extends non-parallel to the central axis O of the inner layer tube 1.

The outer circumferential surface 1b together with the groove 1c are covered by an outer layer portion L1 described later such that the outer circumferential surface 1b does not have to be a smooth surface. However, in the example shown in FIG. 2, the outer circumferential surface 1b is a smooth cylindrical surface coaxial with the inner circumferential surface 1a.

The shape of the groove 1c is not particularly limited as long as the flexibility of the inner layer tube 1 may be improved.

For example, the groove 1c may be a single spiral groove or a multi-row spiral groove. For example, the groove 1c may be formed in a mesh shape in which a plurality of spiral grooves having different turning directions or turning angles intersect with each other. Further, the groove 1c may be formed discontinuously.

The cross-sectional shape of the groove 1c is also not particularly limited. For example, the cross-sectional shape of the groove 1c may be a C-shape or a U-shape such as a semicircular shape or a semi-elliptical shape, a triangular shape (V-shape), a rectangular shape, a polygonal shape, or the like.

In the example shown in FIG. 2, the groove 1c is a single spiral groove that swirls along the outer circumferential surface 1b. The cross-sectional shape of the groove 1c is a C-shape formed by an arc having a central angle equal to or less than 180 degrees.

The groove width, depth, and swivel pitch of the groove 1c are not particularly limited as long as the necessary flexibility may be provided to the inner layer tube 1.

For example, the groove width of the groove 1c may be equal to or more than 0.2 mm and equal to or less than 2 mm. The groove width of the groove 1c is more preferably to be equal to or more than 0.3 mm and equal to or leas than 0.6 mm.

For example, the depth of the groove 1c may be equal to or more than 0.05 mm and equal to or less than 0.5 mm. The depth of the groove 1c is more preferably to be equal to or more than 0.15 mm and equal to or less than 0.3 mm.

For example, the turning pitch of the groove 1c may be equal to or more than 1 mm and equal to or less than 20 mm. The turning pitch of the groove 1c is more preferably equal to or more than 2 mm and equal to or less than 10 mm.

The outer circumferential surface 1b and the groove 1c may be surface processed so as to improve the adhesion with the outer layer portion L1 described later. For example, a chemical etching treatment with a metallic sodium solution or the like, a treatment by plasma irradiation, a polishing treatment by machining, or the like may be applied to the outer circumferential surface 1b and the groove 1c.

A suitable resin for achieving the necessary flexibility required for the inner layer tube 1 is used as the material of the inner layer tube 1. It is more preferable that a resin having superior slipperiness is used as the material of the inner layer tube 1 so as to suppress the wear on the inner circumferential surface 1a.

It is more preferable that a resin material having superior chemical resistance, biocompatibility, cleaning and disinfecting property, airtightness, liquid tightness, and the like is used as the material of the inner layer tube 1 depending on the necessity of the medical device in which the inner layer tube 1 is used.

As the material of the inner layer tube 1, for example, the general purpose plastic such as polyethylene, polypropylene, polystyrene, and polyvinyl chloride may be used.

As the material of the inner layer tube 1, for example, the engineering plastic such as polycarbonate, polyacetal, and polyamide may be used.

As the material of the inner layer tube 1, for example, the super engineering plastic such as polysulfone and polyimide polyether nitrile may be used.

As the material of the inner layer tube 1, for example, the fluororesin such as polytetrafluoroethylene (PTFE), ethylene-tetrafluoroethylene copolymer, and tetrafluoroethylene-hexafluoropropylene copolymer may be used.

As the material of the inner layer tube 1, for example, the thermoplastic elastomer such as a fluorine-based thermoplastic elastomer and the like may be used.

Each of the above-described materials may be individually used in the inner layer tube 1, or may be used as a composite material in which a plurality of the above-described materials are combined. In a case in which a composite material is used in the inner layer tube 1, the composite material may be a material in which a plurality of materials are dispersed and blended. In a case in which a composite material is used for the inner layer tube 1, the plurality of materials may have a layered structure.

Among the above-described materials, the inner layer tube 1 is more preferably to be formed from a non-porous fluororesin since the non-porous fluororesin has excellent chemical resistance to chemicals used for sterilization and the like. The non-porous fluororesin is also suitable in biocompatibility, cleaning and disinfecting properties, airtightness, and liquid tightness.

Further, the fluororesin is also suitable in slipperiness such that the frictional force against the rigid member such as the treatment tool is reduced. As a result, the kink resistance is further improved in that the wear amount on the inner circumferential surface 1a is reduced.

Among the fluororesins, PTFE is particularly preferable since PTFE has particularly suitable chemical resistance.

The outer layer portion L1 is a tubular layered portion that surrounds the groove 1c and the outer circumferential surface 1b of the inner layer tube 1. The outer layer portion L1 has an elastomer layer 2 formed from an elastomer resin that is softer than the resin material of the inner layer tube 1.

The elastomer layer 2 fills the groove 1c of the inner layer tube 1 while covering the outer circumferential surface 1b (outer circumference) of the inner layer tube 1. The inner surface 2a of the elastomer layer 2 is in close contact with the outer circumferential surface 1b and the groove 1c.

The outer circumferential surface 2b of the elastomer layer 2 has a cylindrical surface shape coaxial with the central axis O.

The material of the elastomer layer 2 is not particularly limited as long as being softer than the resin material (base material) of the inner layer tube 1. Here, the degree of softness is defined by the magnitude of the elastic modulus of the resin. In other words, an elastomer resin having an elastic modulus smaller than that of the resin material of the inner layer tube 1 is used in the material of the elastomer layer 2.

As the material of the elastomer layer 2, for example, a thermoplastic elastomer such as a urethane-based thermoplastic elastomer may be used.

As the material of the elastomer layer 2, for example, vulcanized rubber such as isoprene rubber, butyl rubber, ethylene-propylene rubber, chloroprene rubber, nitrile rubber, silicone rubber, urethane rubber, and fluororubber may be used.

Each of the above-described materials may be individually used for the elastomer layer 2, or may be used as a composite material in which a plurality of the above-described materials are combined. In a case in which a composite material is used for the elastomer layer 2, the composite material may be a material in which a plurality of materials are dispersed and blended. In a case in which a composite material is used for the elastomer layer 2, the plurality of materials may have a layered structure.

As the material of the elastomer layer 2, a porous body or a foam formed from the above-described material or composite material may be used. In this case, the flexibility of the channel tube 10 may be further improved.

When a plurality of materials are used in the elastomer layer 2, different materials may be used in the longitudinal direction. In this case, the characteristic at each position in the longitudinal direction of the channel tube 10 can be changed according to the difference in material characteristics.

For example, as the material of the elastomer layer 2, materials having different modulus of longitudinal elasticity in the longitudinal direction may be used. In this case, the flexibility of the channel tube 20 can be changed in the longitudinal direction.

For example, as the material of the elastomer layer 2, the vulcanized rubber may be used at the portion inserted through the bending portion 103, and the thermoplastic elastomer may be used at the other portions. In this case, in the bending portion 103 where the bending load is applied, the vulcanized rubber having suitable bending resistance and extending characteristics is used such that the durability and flexibility can be improved. Since the thermoplastic elastomer having high rigidness is used for other portions, the insertability of the endoscope can be improved.

As the elastomer layer 2, among the above-described materials, a particularly preferable material is a peroxide-crosslinked rubber or a thermoplastic elastomer in which a peroxide-crosslinked rubber is dispersed. As the peroxide crosslink, an organic peroxide crosslink is more preferable.

Specific examples of such particularly preferable materials include, for example, peroxide-crosslinked fiuororubber, polyurethane elastomer in which particles of silicone rubber are dispersed and the like.

The peroxide-crosslinked rubber or the thermoplastic elastomer in which the peroxide-crosslinked rubber is dispersed is excellent in softness while hardly adhering to the metal braid 3 described later. As a result, the elasticity of the outer layer portion L1 is improved. Accordingly, the flexibility of the channel tube 10 is further improved.

Additives other than the elastomer resin may be added to the inside of the elastomer layer 2 as necessity. For example, carbon, silica, alumina, and the like may be added to the elastomer layer 2.

A metal braid 3 (braid) is arranged inside the elastomer layer 2. However, the metal braid 3 is arranged only in a place other than the groove 1c inside the outer layer portion L1. In other words, the metal braid 3 is not included inside the elastomer layer 2 that fills the groove 1c. Specifically, the metal braid 3 is arranged between the outer circumferential surface 1b and the outer circumferential surface 2b in the layer thickness direction of the elastomer layer 2.

As an example, the metal braid 3 shown in FIG. 2 is arranged to abut on the outer circumferential surface 1b. However, a layered portion composed from the elastomer layer 2 only may be formed at least in a portion between the metal braid 3 and the outer circumferential surface 1b.

The metal braid 3 is used to reinforce the channel tube 10.

The metal braid 3 is a net-shaped body formed from metal strand wires (metal wires).

The shape of the metal strand wire is not particularly limited. Examples of the shape of the strand wire include a round wire, a flat wire, a stranded wire and the like. For example, the bold (thickness) of the metal wire in the thickness direction of the metal braid 3 may be equal to or larger than 0.03 mm and equal to or less than 0.3 mm. The bold (thickness) of the metal wire is more preferably equal to or more than 0.05 mm and equal to or less than 0.15 mm.

The metal strand wires used in the metal braid 3 may be a single type of wire, or may be a combination of a plurality of typos of strand wires at least having either of different materials or shapes. In a case in which a plurality of types of strand wires are used in the metal braid 3, they may be twisted together or may be arranged at different positions.

For example, the metal braid 3 may be a net-shaped body braided by the metal strand wires or a net-shaped body woven by the metal strand wires. The method of braiding the metal strand wires and the method of weaving the metal strand wires are not particularly limited as long as the necessary strength and flexibility required for the metal braid 3 can be obtained. Examples of the braiding method and the weaving method of the net-shaped body include plain weave, twill weave, satin weave, knotless net and the like.

In the example shown in FIG. 2, the metal braid 3 is formed from a cylindrical net-shaped body by the twill weave such that every two metal strands intersect with each other.

The metal braid 3 is made from a net-shaped body such that there is a gap between the metal strand wires that communicates with the metal braid 3 in the thickness direction (diameter direction in the channel tube 10).

The metal braid 3 is embedded inside the elastomer layer 2. Accordingly, the elastomer layer 2 penetrates into the gap of the metal braid 3. The elastomer layer 2 forms a continuous layered portion from the outer circumferential surface 2b to the outer circumferential surface 1b and the groove 1c, except for the portion excluded by the metal braid 3.

With such a configuration, the metal braid 3 is integrated with the elastomer layer 2 inside the elastomer layer 2.

Examples of the material of the metal wire used for the metal braid 3 include copper, copper alloy, piano wire, stainless steel, titanium, titanium alloy, nickel titanium alloy, tungsten, tungsten alloy, nickel alloy, cobalt alloy, amorphous metal and the like.

The material of the metal wire is more preferably a metal having suitable tenacity and being difficult to be corroded by the autoclave sterilization. Examples of metals that are particularly suitable in tenacity and corrosion resistance include the stainless steel.

Next, a method of manufacturing the channel tube 10 will be described.

First, the inner layer tube 1 having the groove 1c is prepared.

The groove 1c may be formed at the time of molding the inner layer tube 1, or the groove 1c may be formed by a removal process after the cylindrical tube to form the inner layer tube 1 is manufactured.

Thereafter, the metal braid 3 is laminated around the circumference of the outer circumferential surface 1b of the inner layer tube 1. Thereafter, the elastomer layer 2 is formed so as to cover the metal braid 3.

For example, the extrusion molding may be used to form the elastomer layer 2. The elastomer layer 2 comes into close contact with the outer circumferential surface 1b and the surface of the groove 1c of the inner layer tube 1 through the net-shaped gaps of the metal braid 3.

As a result, the metal braid 3 is embedded inside the elastomer layer 2 such that the outer layer portion L1 is formed.

In this manner, the channel tube 10 is manufactured.

The effects of the channel tube 10 will be described.

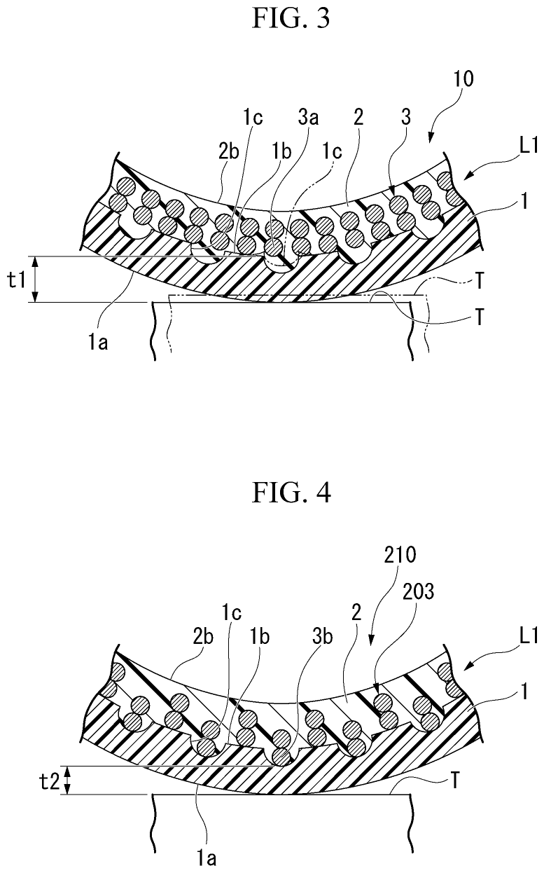

FIG. 3 is a schematic view showing the effects of the medical device tube according to the first embodiment of the present disclosure.

The groove 1c that is non-parallel to the central axis O is formed on the outer circumference of the inner layer tube 1. When the cross section including the central axis O is taken, the cross section of the groove crossing the groove 1c is shown at a position separated in the longitudinal direction of the inner layer tube 1. As shown in the schematic cross section inward the bending in FIG. 3, when the inner layer tube 1 is bent in the direction of bending the central axis O, each groove 1c is deformed toward the direction where the groove width of each groove 1c is reduced on the surface at the inward side of the bending where the bending stress in the compression direction is maximized. Since the layer thickness of the inner layer tube 1 is thin at the portion where each groove 1c is formed, the inner layer tube 1 is bent from the groove bottom of each groove 1c at the inward side of the bending. Although not particularly shown in figures, on the surface at the outward side of the bending, the groove width of each groove 1c at the outward side of the bending is enlarged by the bending stress in the tensile direction, and the surface is bent from the bottom of each groove 1c.

In this manner, the whole inner layer tube 1 is bent in a bow shape.

In a case in which the grooves 1c are formed evenly in the circumferential direction and the axial direction (longitudinal direction) of the inner layer tube 1, the flexibility of the inner layer tube 1 is equalized in the circumferential direction and the axial direction.

In the channel tube 10, each groove 1c is partially filled with the elastomer layer 2. Accordingly, the inner layer tube 1 becomes difficult to be bent to some level as compared with the individual inner layer tube 1. However, since the elastomer layer 2 is softer than the material of the inner layer tube 1, it is impossible for the elastomer layer 2 to hinder the bending of the grooves 1c such that the inner layer tube 1 does not bend. As a result, better flexibility can be achieved as compared with the case in which the elastomer layer 2 is laminated on the inner layer tube without the groove 1c.

Further, since the elastomer resin of the elastomer layer 2 is filled in the groove 1c, it is possible to prevent the groove 1c from being completely crushed in the groove width direction. Accordingly, there is no buckling to be induced due to the crush of the groove 1c such that the deformation in a V-shape with an acute angle is prevented.

For example, in the channel tube 10, when an inner layer tube without the groove 1c is used instead of the inner layer tube 1, the inner layer tube has higher rigidity than the inner layer tube 1. Accordingly, the flexibility of the channel tube using such an inner layer tube is inferior than the flexibility of the channel tube 10.

In this case, it is considerable to reduce the thickness of the inner layer tube so as to ensure the flexibility. However, if the thickness of the inner layer tube is reduced, a margin for wear of the inner circumferential surface of the inner layer tube is reduced such that the durability of the inner layer tube may be lowered.

For example, in a case in which a groove is provided on the outer circumferential surface of the inner layer tube to improve the flexibility, it is also considerable to form the groove extending parallelly to the central axis O in the inner layer tube. In this case, since the inner layer tube is easily bent due to the decrease in the moment of inertia of area of the inner layer tube, the flexibility is increased as compared with the case in which the similar groove is not provided.

However, if the groove extends in a direction orthogonal to the bending direction, a locally low-rigidity portion which is easily bent at the time of bending the inner layer tube is not formed. In this case, in order to achieve the same flexibility as in the present embodiment, it is necessary to uniformly reduce the flexural rigidity of the inner layer tube along the longitudinal direction such that it is necessary to form a large number of grooves in the circumferential direction or a groove deeper than the groove 1c. As a result, the whole inner layer tube becomes low rigidity and a number of the partially thin portions increases such that the durability may be reduced.

In the channel tube 10, an outer layer portion L1 including a rigid metal braid 3 is laminated on the outer circumference of the inner layer tube 1.

Since the elastomer layer 2 of the outer layer portion L1 is in close contact with the outer circumferential surface 1b and the groove 1c of the inner layer tube 1, for example, when the inner layer tube 1 receives an external, force so as to be deformed, the external force is also transmitted from the inner layer tube 1 to the outer layer portion L1. The outer layer portion L1 is deformed in the same manner as the inner layer tube 1.

Since the elastomer layer 2 is formed from an elastomer resin softer than the inner layer tube 1, it is easily deformed together with the inner layer tube 1.

Since the metal braid 3 is made of a net-shaped body, the metal braid 3 has the flexibility such that a shape of the mesh changes due to the deformation of the outer layer portion L1. Further, the metal braid 3 has the elasticity in the direction along the central axis O of the inner layer tube 1 due to the shape change or the mesh.

Since the metal braid 3 is formed from metal wires that is more rigid than the material of the inner layer tube 1, the metal braid 3 has the shape retainability so as to maintain the tubular shape against the external force. As a result, the metal braid 3 functions as a reinforcing member that suppresses the deformation of the inner layer tube 1 integrated via the elastomer layer 2.

For example, the metal braid 3 becomes a member that resists the crushing of the inner circumferential surface 1a of the inner layer tube 1 in a case in which the external force for crushing the inner layer tube 1 in the radial direction is applied or the channel tube 10 is bent.

In this manner, the channel tube 10 is reinforced by the outer layer portion L1 without impairing the flexibility.

According to the channel tube 10, since the outer layer portion L1 has the metal braid 3 having the shape retainability, the kink resistance is further improved. Further, since the metal braid 3 easily expands and contracts in the direction along the central axis O, the resistance to the bending that is received by the channel tube 10 is reduced. As a result, the flexibility of the channel tube 10 is further improved.

Further, in the channel tube 10, the metal braid 3 is arranged only in the locations other than the groove 1c. The effects of such configuration will be described in comparison with the comparison example shown in FIG. 4.

FIG. 4 is a schematic view showing the effects of the tube for a medical device according to the comparison example.

The channel tube 210 according to the comparison example includes a metal braid 203 instead of the metal braid 3 in the outer layer portion L1 of the channel tube 10.

Similar to the metal braid 3, the metal braid 203 is made of a net-shaped body formed from metal strand wires. However, a part of the metal strand wires of the metal braid 203 has entered the inward side of the groove 1c together with the elastomer layer 2. In the example shown in FIG. 4, one metal strand wire in the metal braid 203 has entered to the vicinity of the groove bottom of the groove 1c.

In this case, both the rigid metal strand wires and the soft elastomer layer 2 are filled in the groove 1c of the channel tube 210. Since the elastomer layer 2 is only excluded by the volume of the metal wire, the deformable amount of the groove 1c is lower than the deformable amount of the groove 1c in the channel tube 10. As a result, the flexibility of the channel tube 210 is lower than that of the channel tube 10.

Further, since the elastomer layer 2 that enters between the groove bottom of the groove 1c and the metal strand wires filled in the groove 1c is also reduced, the cushioning characteristic of the elastomer layer 2 between the metal strand wires and the inner layer tube 1 is also reduced.

As shown in FIG. 3 and FIG. 4, when the treatment tool T is inserted into each of the channel tubes 10 and 210 in the bent state, the treatment tool T abuts or slides on the convex portion of the inner circumferential surface 1a.

As shown in FIG. 3, in the channel tube 10, the metal braid 3 and the treatment tool T are configured to sandwich the inner layer tube 1 and the elastomer layer 2 therebetween and separated from each other by equal to or more than the thickness t1 of the inner layer tube 1.

Particularly, at the rear side of the apex portion of the bending of the inner layer tube 1, the metal strand wire 3a that is the closest to the treatment tool T is opposite to the treatment tool T with the elastomer layer 2 filled in the groove 1c sandwiched therebetween.

Accordingly, when the inner layer tube 1 is pressed against the metal strand wire 3a by the external force received from the treatment tool T, the stress transmitted to the metal strand wire 3a is reduced due to the deformation of the elastomer layer 2 in the groove 1c. For example, when the groove 1c is deformed toward the metal braid 3 as shown by the two-dot chain wire, the external force transmitted to the metal strand wire 3a and the reaction to the treatment tool T are reduced by crushing the elastomer layer 2. As described above, the soft elastomer layer 2 in the groove 1c functions as a cushioning material (cushion).

As a result, the sliding friction between the treatment tool T and the inner circumferential surface 1a is reduced, such that the progress of wear of the inner circumferential surface 1a is suppressed.

Further, even if the wear in the inner circumferential surface 1a progresses, the metal strand wire 3a is not exposed until the inner circumferential surface 1a is worn by the thickness t1, such that the sliding characteristic with the treatment tool T is maintained.

On the other hand, as shown in FIG. 4, in the channel tube 210 of the comparison example, the metal strand wire 3b having the shortest distance between the metal braid 203 and the treatment tool T is separated from the treatment tool T by a distance t2 (however, t2<t1). The metal strand wire 3b is opposite to the treatment tool T with the inner layer tube 1 from the inner circumferential surface 1a to the groove bottom of the groove 1c interposed therebetween rather than the elastomer layer 2.

The inner layer tube 1 is more rigid than the elastomer layer 2 and has a lower cushioning characteristic. Accordingly, the external force received from the treatment tool T is more easily transmitted to the metal strand wire 3b than when the elastomer layer 2 is interposed. Accordingly, since the reaction from the metal strand wire 3b becomes large, the sliding friction of the treatment tool T becomes large as compared with the channel tube 10, and the wear of the inner circumferential surface 1a is accelerated.

Further, since the inner circumferential surface 1a has only the thickness t2 as a margin to withstand wear, the metal strand wire 3b is exposed in a shorter time than that of the channel tube 10. As a result, the sliding characteristics with the treatment tool T deteriorate.

As described above, according to the channel tube 10 according to the present embodiment, it is possible to reduce wear on the inner circumferential portion while maintaining the flexibility and the kink resistance. Further, according to the endoscope 100 of the present embodiment, the durability can be improved by including the channel tube 10.

### Second Embodiment

Next, a tube for a medical device tube according to a second embodiment of the present disclosure will be described.

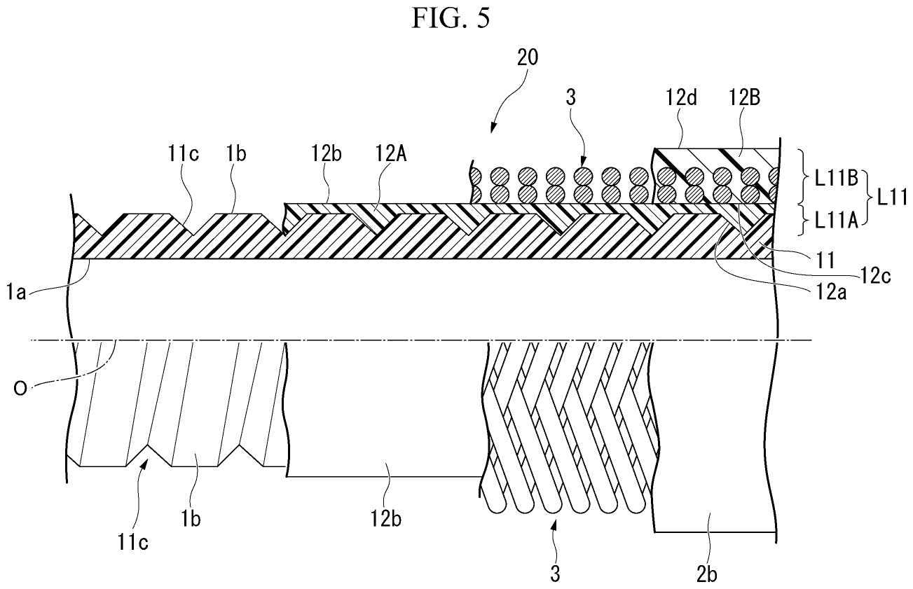

FIG. 5 is a schematic partial cross-sectional view showing a configuration example of the tube for a medical device tube according to the second embodiment of the present disclosure.

As shown in FIG. 1, a channel tube 20 (a tube for a medical device) of the present embodiment can be used in the endoscope 100 according to the first embodiment instead of the channel tube 10 according to the first embodiment.

As shown in FIG. 5, the channel tube 20 includes an inner-layer tube 11 and an outer layer portion L11 (outer layer) instead of the inner layer tube 1 and the outer layer portion L1 of the medical device tube 10 according to the first embodiment.

Hereinafter, the differences from the first embodiment, will be mainly focused and described.

The inner layer tube 11 includes a groove 11c instead of the groove 1c of the inner layer tube 1 according to the first embodiment. The groove 11c is formed of a single spiral groove similar to the groove 1c except for having a V-shaped cross section.

The material of the inner layer tube 11 can be selected from the materials suitable for the inner layer tube 1 according to the first embodiment.

The outer layer portion L11 is formed from an elastomer resin that is softer than the resin material (base material) of the inner layer tube 11. The outer layer portion L11 is divided into at least two layers, and the outer layer portion L11 includes a buffer layer L11A (the innermost layer in the outer layer) and a reinforcing layer L11B (a layer other than the innermost layer in the outer layer). In the example shown in FIG. 5, the outer layer portion L1 is formed from the buffer layer L11A and the reinforcing layer L11B. For example, one or more layers formed from an elastomer resin softer than the resin material of the inner layer tube 11 may be included between the buffer layer L11A and the reinforcing layer L11B, or on the reinforcing layer L11B.

The buffer layer L11A is a tubular layered portion that surrounds the groove 11c and the outer circumferential surface 1b of the inner layer tube 11. The buffer layer L11A has an elastomer layer 12A formed from an elastomer resin that is softer than the resin material of the inner layer tube 11. However, the metal braid 3 is not included inside the buffer layer L11A.

The elastomer layer 12A fills the groove 11c of the inner layer tube 11 and covers the outer circumferential surface 1b (outer circumference). The inner surface 12a of the elastomer layer 12A is in close contact with the outer circumferential surface 1b and the groove 11c.

The outer circumferential surface 12b of the elastomer layer 12A has a cylindrical surface shape to be coaxial with the central axis O.

The material of the elastomer layer 12A can be selected from the materials suitable for the elastomer layer 2 according to the first embodiment. The material of the elastomer layer 12A may be the same as the material of the elastomer layer 2 according to the first embodiment or different from the material of the elastomer layer 2 according to the first embodiment.

However, it is mere preferable that the elastomer layer 12A is formed from a softer material than that of the elastomer layer 12B described below.

The reinforcing layer L11B is a tubular layered portion that surrounds the buffer layer L11A from the outside. One or more intermediate layers made of an elastomer resin may be interposed between the reinforcing layer L11B and the buffer layer L11A. Further, one or more outer layers formed from an elastomer resin may be laminated on the outside of the reinforcing layer L11B.

In the example shown in FIG. 5, the reinforcing layer L11B is closely laminated on the outer circumferential surface 12b of the buffer layer L11A, and the reinforcing layer L11B forms the outermost layer of the channel tube 20.

The reinforcing layer L11B has an elastomer layer 12B formed from an elastomer resin that is softer than the resin material of the inner layer tube 11. The inner peripheral surface 12c of the elastomer layer 12B is in close contact with the outer circumferential surface 12b of the elastomer layer 12A. The outer circumferential surface 12d of the elastomer layer 12B has a cylindrical surface shape coaxial with the central axis O.

The material of the elastomer layer 12B can be selected from the materials suitable for the elastomer layer 2 in the first embodiment. The material of the elastomer layer 12B only has to be different from the material of the elastomer layer 12A, and the material of the elastomer layer 12B may be the same as the material of the elastomer layer 2 according to the first embodiment or different from the material of the elastomer layer 2 according to the first embodiment.

However, it is more preferable that the elastomer layer 12B is formed from a more rigid material than that of the elastomer layer 12A.

The metal braid 3 similar to the first embodiment is embedded in the elastomer layer 12B. Accordingly, the elastomer-layer 12B penetrates into the gap of the metal braid 3. The elastomer layer 12B forms a continuous layered portion from the outer circumferential surface 12d to the inner circumferential surface 12c except for the portion excluded by the metal braid 3.

With such a configuration, the metal braid 3 is integrated with the elastomer layer 12B inside the elastomer layer 12B.

As an example, the metal braid 3 shown in FIG. 5 is arranged to abut on the outer circumferential surface 12b of the elastomer layer 12A. However, a layered portion composed from only the elastomer layer 12B may be formed at least in a part between the metal braid 3 and the outer circumferential surface 12b.

Since the metal braid 3 according to the present embodiment, is arranged only inside the reinforcing layer L11B, the metal braid 3 is arranged only at a location other than the groove 11c inside the outer layer portion L11.

In order to manufacture such a channel tube 20, the inner layer tube 11 is prepared as in the first embodiment. Thereafter, for example, the buffer layer L11A is formed on the outer circumferential surface 11b and the groove 11c surface of the inner layer tube 11 by the extrusion molding.

Thereafter, the elastomer layer 123 is formed by extrusion molding with the metal braid 3 arranged on the outer circumferential surface 12b.

In this manner, the channel tube 20 is manufactured.

According to the channel tube 20, the configuration that the cross-sectional shape of the groove 11c in the inner layer tube 11 is V-shaped is different from the groove 1c in which the cross-sectional shape according to the first embodiment is not particularly limited. According to the channel tube 20, the configuration that the metal braid 3 is arranged in the reinforcing layer L11B with the buffer layer L11A sandwiched therebetween is different from the configuration in which the metal braid 3 is arranged only at locations other than the groove 1c in the outer layer portion L1 of the elastomer layer 2 according to the first embodiment described above. Further, according to the channel tube 20, since the materials of the elastomer layers 12A and 12B are different from each other, the configuration that the outer layer portion L11 is composed from at least two layers is different from the outer layer portion L1 having the elastomer layer 2 only according to the first embodiment described above.

However, even if the groove shape of the groove 11c is V-shaped, the inner layer tube 11 having the same flexibility as the inner layer tube 1 may be formed by appropriately adjusting the groove width, depth, swivel pitch and the like. Moreover, the groove 11c is filled with the elastomer layer 12A which is softer than the material of the inner layer tube 11, and the reinforcing layer L11B having the metal braid 3 is laminated on the outer side of the buffer layer L11A.

According to the channel tube 20 of the present embodiment, it is possible to reduce the wear of the inner peripheral portion while maintaining the flexibility and the kink resistance as in the first embodiment.

Particularly, in the present embodiment, since the elastomer layer 12A is also arranged on the outer circumferential surface 1b of the inner layer tube 11, the cushioning characteristic due to the deformation of the elastomer layer 12A is also applied to the outer circumferential surface 1b. Accordingly, in addition to the metal strand wires on the groove 11c, the external force from the treatment tool becomes difficult to be transmitted to the metal strand wires on the outer circumferential surface 1b. As a result, the wear of the inner circumferential surface 1a can be further reduced.

According to the channel tube 20, since the buffer layer L11A is provided, the external force applied to the reinforcing layer L11B from the outside is dispersed by the metal braid 3, and then further dispersed due to the cushioning characteristic of the elastomer layer 12A while the stress is relaxed. Accordingly, the pressing force applied to the channel tube 20 from the outside is also difficult to be transmitted to the inner layer tube 11. Accordingly, even in a case in which the treatment tool is slid while the pressing force is applied from the outside of the channel tube 20, local wear due to the rigid member such as the treatment tool sliding on the inner circumferential surface 1a can be reduced.

Further, in the channel tube 20, different types of resins can be used in the elastomer resin that fills the groove 11c and the elastomer resin in which the metal braid 3 is arranged. Accordingly, for example, the elastomer layer 12A may be formed by using a softer material so as to improve the cushioning effect. The elastomer layer 12B may be formed by using a more rigid material so as to improve the strength of the outermost strength portion.

Further, since the channel tube 20 is manufactured by arranging the metal braid 3 after covering the outer circumference of the inner layer tube 11 with the elastomer layer 12A, it is possible to prevent part of the metal braid 3 from being arranged in the groove 11c due to the production tolerance or the like.

### First Modification

A tube for a medical device according to a modification (first modification) of the second embodiment of the present disclosure will be described.

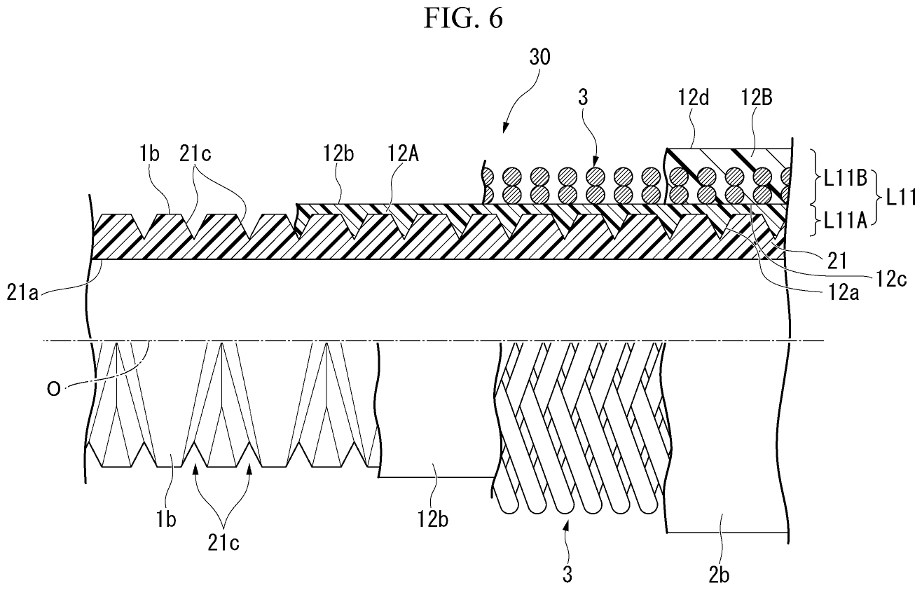

FIG. 6 is a schematic partial cross-sectional view showing a configuration example of a tube for a medical device according to a modification (first modification) of the second embodiment of the present disclosure.

As shown in FIG. 1, a channel tube 30 (a tube for a medical device) according to the present modification can be used in the endoscope 100 according to the first embodiment instead of the channel tube 10 according to the first embodiment.

As shown in FIG. 6, the channel tube 30 includes an inner layer tube 21 instead of the inner layer tube 11 of the tube for a medical device 20 according to the second embodiment.

Hereinafter, the differences between the present modification and the second embodiment will be mainly described.

The inner layer tube 21 includes a groove 21c instead of the groove 11c of the inner layer tube 11 according to the second embodiment. The groove 21c is formed in a mesh shape formed by intersecting two spiral grooves having different turning directions. The cross-sectional shape of the groove 21c is V-shaped.

The turning angles of the two spiral grooves may be different from each other; however, the example shown in FIG. 6, the turning angles are equal to each other. The intersecting position of each spiral groove is on a straight line parallel to the central axis O facing each other in the radial direction. However, the intersection position of each spiral groove is not limited thereto.

Since the channel tube 30 is different from the channel tube 20 only in the shape of the groove 21c in the inner layer tube 21, the channel tube 30 can be manufactured in the same manner as in the second embodiment except that, the inner layer tube 21 is prepared instead of the inner layer tube 11.

According to the channel tube 30, the same effects as that of the channel tube 20 according to the second embodiment are achieved except for the shape of the groove 21c.

According to the channel tube 30 of the present modification, it is possible to reduce the wear of the inner peripheral portion while maintaining the flexibility and the kink resistance as in the second embodiment.

According to the first embodiment, the example in which the metal braid 3 is arranged on the outer circumferential surface 1b of the inner layer tube 1 has been described. However, in the first embodiment, a layered portion of the elastomer layer 2 can be formed only between the outer circumferential surface 1b and the metal braid 3. For example, the elastomer layer 2 may be formed in a state in which the metal braid 3 is arranged to be spaced away from the outer circumferential surface 1b, or the elastomer layer 2 may be formed to be divided into two layers in the same manner as in the second embodiment.

According to such a configuration, since the buffer layer formed from the elastomer layer 2 is formed only between the metal braid 3 and the outer circumferential surface 1b, the same effects as the second embodiment are achieved due to the cushioning characteristic of the elastomer layer 2 between the metal braid 3 and the outer circumferential surface 1b as in the second embodiment.

In the description of the second embodiment, the case where the buffer layer L11A fills the groove 11c and covers the outer circumferential surface 1b has been described. However, the buffer layer L11A may only fill the groove 11c without covering the outer circumferential surface 1b. In this case, the configuration is substantially the same as that of the first embodiment; however, since the types of materials between the elastomer resin that fills the groove 11c and the elastomer resin including the metal braid 3 are different from each other, the elastomer to be used can be optimized depending on each usage.

### EXAMPLES

Next, Examples 1 to 3 of the tube for a medical device corresponding to the above-described first embodiment, second embodiment and the first modification will be described together with Comparison examples 1 and 2.

### Example 1

Example 1 is an example corresponding to the channel tube 10 (see FIG. 1) according to the above-described first embodiment.

The inner layer tube 1 in Example 1 was formed by forming a groove 1c composed of a single spiral groove on the outer circumferential surface 1b of a cylindrical tube having an inner diameter of 3.2 mm and a thickness of 0.4 mm.

The cross-sectional shape of the groove 1c was a semicircle with a radius of 0.2 mm. The turning pitch of the groove 1c was set to 0.8 mm.

Polytetrafluoroethylene was used as the material of the inner layer tube 1.

The thickness of the outer layer portion L1 from the outer circumferential surface 1b was set to 0.3 mm. Fluororubber was used as the material of the outer layer portion L1.

The metal braid 3 was formed by twill weaving a piano wire having a diameter of 0.1 mm. The conditions for weaving the metal braid 3 were set to as 1 wire in a group, 16 groups and 30 PPI.

The channel tube 10 of Example 1 was manufactured as follows.

After the inner layer tube 1 was prepared, the inner layer tube 1 was surface-treated by using the plasma irradiation. Thereafter, in a state in which the metal braid 3 is arranged on the outer circumferential portion of the inner layer tube 1, the metal braid 3 was coated by extrusion molding with fluororubber such that the layer thickness was 0.3 mm. The metal braid 3 was arranged outside the outer circumferential surface 1b without entering the groove 1c.

### Example 2

Example 2 is an example corresponding to the channel tube 20 (see FIG. 2) according to the second embodiment.

The inner layer tube 11 in Example 2 is configured by forming the groove 11c on the outer circumferential surface 1b of the same cylindrical tube as in Example 1.

The cross-sectional shape of the groove 11c was an isosceles triangle having an opening width of 0.4 mm and a depth of 0.2 mm. The turning pitch of the groove 11c was set to 0.8 mm as same as in Example 1.

The outer diameter of the outer circumferential surface 11b of the buffer layer L11A was set to 4.2 mm. Silicone rubber was used as the material of the buffer layer L11A.

The reinforcing layer L11B was configured in the same manner as the outer layer portion L1 of Example 1 except that the reinforcing layer L11B was laminated on the outer circumferential surface 11b.

In the channel tube 20 of Example 2 was manufactured in the same manner as Example 1 except that the inner layer tube 11 was used instead of the inner layer tube 1, and after the buffer layer L11A was formed on the inner layer tube 11 by extrusion molding, the reinforcing layer L11B was formed in the same manner as in the first embodiment.

### Example 3

Example 3 is an example corresponding to the channel tube 30 (see FIG. 3) of the first modification of the second embodiment. The channel tube 30 of Example 3 was configured in the same manner as in Example 2 except that the inner layer tube 21 having the groove 21c was used instead of the inner layer tube 11.

The cross-sectional shape of the groove 21c was an isosceles triangle having an opening width of 0.2 mm and a depth of 0.2 mm. The groove 21c was formed of two spiral grooves having a turning pitch of 0.8 mm and rotating in opposite directions to each other.

The channel tube 30 of Example 3 was manufactured in the same manner as in Example 2 except that the inner layer tube 21 was used instead of the inner layer tube 11.

### Comparison Example 1

Comparison example 1 is an example in which the groove 1c is not formed in the configuration shown in Example 1. Hereinafter, the differences from Example 1 will be mainly described.

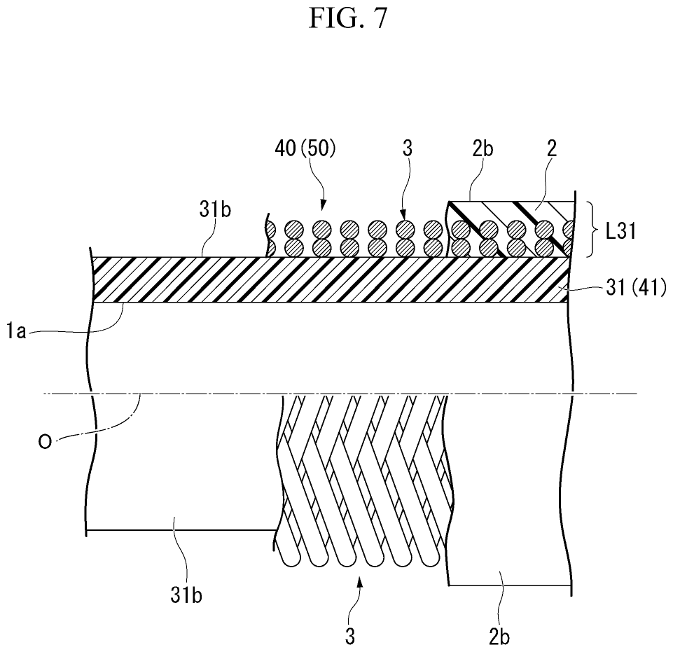

FIG. 7 is a schematic partial cross-sectional view showing a configuration example of the tube for a medical device tube of Comparison example 1.

As shown in FIG. 7, the channel tube 40 of Comparison example 1 includes an inner layer tube 31 instead of the inner layer tube 1 of Example 1.

The inner layer tube 31 was a cylindrical tube having an inner diameter of 3.2 mm and a thickness of 0.4 mm, and the outer circumferential surface 31b of the inner layer tube 31 was a smooth cylindrical surface. Accordingly, the outer layer portion L31 (outer layer) in the present example includes an elastomer layer 2 formed from fluororubber having a thickness of 0.3 mm from the outer circumferential surface 31b, and the metal braid 3 same as that in Example 1 arranged on the outer circumferential surface 31b.

The channel tube 40 of Comparison example 1 was manufactured in the same manner as in Example 1 except that the inner layer tube 31 was used instead of the inner layer tube 1.

### Comparison Example 2

As shown in FIG. 7, the channel tube 50 of Comparison example 2 includes an inner layer tube 41 instead of the inner layer tube 31 of the channel tube 40 of Comparison example 1.

The inner layer tube 41 was configured in the same manner as the inner layer tube 31 of Comparison example 1 except that the thickness was 0.2 mm.

The channel tube 50 of Comparison example 2 was manufactured in the same manner as in Comparison example 1 except that the inner layer tube 41 was used instead of the inner layer tube 31.

### Evaluation

Wear resistance, flexibility, and outer diameter of the channel tube were evaluated using the test samples of the channel tubes of Examples 1 to 3 and Comparison examples 1 and 2.

The evaluation results are shown in [Table 1] below.

TABLE 1

| | EVALUATION RESULT | | |
|---|---|---|---|
| | ABRASION | FLEXI- | COMPREHENSIVE |
| | RESISTANCE | BILITY | EVALUATION |
| EXAMPLE 1 | A | A | A |
| EXAMPLE 2 | AA | A | A |
| EXAMPLE 3 | A | AA | A |
| COMPARISON | A | C | C |
| EXAMPLE 1 | | | |
| COMPARISON | C | A | C |
| EXAMPLE 2 | | | |

### Wear Resistance

It is considerable that the channel tube has a better wear resistance characteristic if the amount of wear on the surface of the inner layer tube due to the insertion and removal of the treatment tool such as forceps is smaller. Accordingly, the amount of wear at the worn part was evaluated after a test in which the forceps were repeatedly inserted and removed from the test samples of the channel tubes in the bending state.

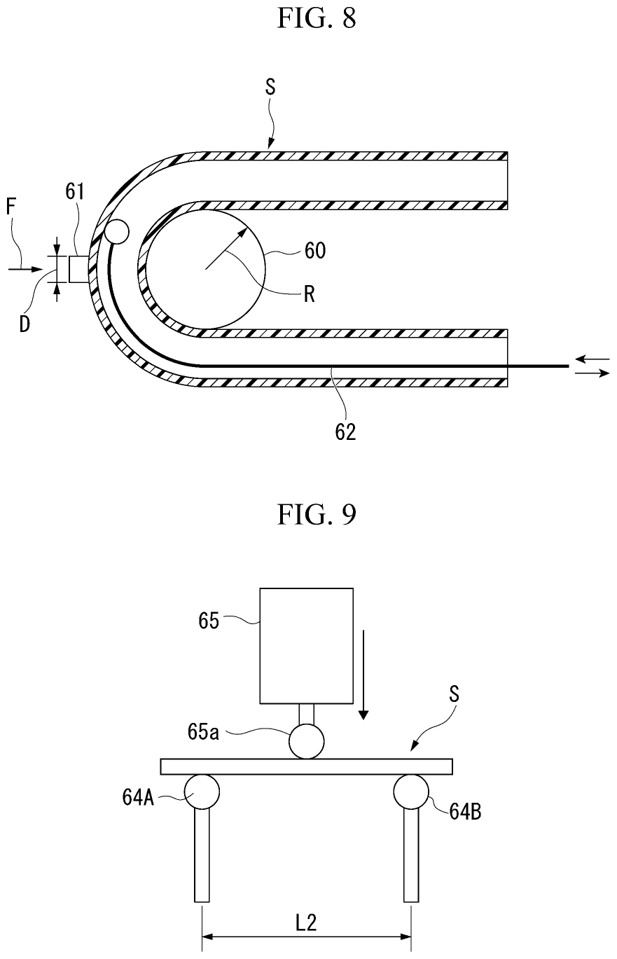

FIG. 8 is a schematic view showing a test method for evaluation of wear resistance.

As shown in FIG. 8, in the wear resistance evaluation, the test sample S was held in a state of being wound for half a circumference and bent by 180 degrees by a cylindrical winding jig 60 having a radius of curvature R=9 (mm). A columnar pressing jig 61 having an outer diameter D=1.6 (mm) was pressed against the bending portion of the test sample S at a pressing force F=1 (N). The pressing jig 61 pressed toward the center of the winding jig 60 at the apex portion of the convex bending portion of the test sample S in a direction parallel to the straight portion of the test sample S.

In such a state, the biopsy forceps 62 was inserted from the end portion of the test sample S. The biopsy forceps 62 was inserted and removed so as to go and return in the range of the bending portion of the test sample S at a speed of 30 mm/sec. As the biopsy forceps 62, FB-25K (product name; manufactured by Olympus Corporation) was used.

Each test sample 5 was inserted and removed for 10,000 times as one round trip of the biopsy forceps 62 was counted as one time. After that, the test sample S was sliced into round slices and the amount of wear of the worn part due to the biopsy forceps 62 was measured using a microscope.

The evaluation criteria for wear resistance was classified as “very good” if the amount of wear is less than 0.01 mm (shown as “AA” (very good) in [Table 1]), “good” if the amount of wear is equal to or more than 0.01 mm and less than 0.05 mm (shown as “A” (good) in [Table 1]), and “poor” if the amount of wear is equal to or more than 0.05 mm (shown as “C” (no good) in [Table 1]).

### Flexibility

The flexibility was evaluated by the amount of pushing force required to bend the test sample S by three-point bending.

FIG. 9 is a schematic view showing a test method for evaluating the flexibility.

As shown in FIG. 9, for the purpose of forming fulcrums at both ends thereof, two pulleys 64A and 64B having a radius of 5 mm were arranged at equal heights with a gap L2=100 (mm) therebetween. The test sample S was placed on the pulleys 64A and 64B. A contact portion 65a of a push-pull gauge 65 was brought into contact with the portion located between the pulleys 64A and 64B from above. The contact portion 65a is provided with a pulley having a radius of 5 mm. The push-pull gauge 65 was pushed downward at a speed of 20 mm/sec with a stroke of 40 mm. At this time, a peak value of the pushing force by the push-pull gauge 65 was measured.

The evaluation criteria was classified as “very good” if the peak value of the pushing force is less than 0.6N (shown as “AA” (very good) in [Table 1]), “good” if the peak value of the pushing force is equal to or more than 0.6N and less than 0.7N (shown as “A” (good) in [Table 1]), and “poor” if the peak value of the pushing force is equal to or more than 0.7N (shown as “C” (no good) in [Table 1]).

### Evaluation Results

As shown in [Table 1], the evaluation results of Examples 1 to 3 were “A” or “AA” for both wear resistance and flexibility, such that the overall evaluation was classified as “good” (shown as “A” (good) in [Table 1]).

Particularly, Example 2 was excellent in terms of wear resistance. According to Example 2, since the buffer layer L11A arranged at the inward side of the metal braid 3 functioned as the cushion layer, it is considerable that the wear resistance was improved as compared with Example 1.

In terms of flexibility, Example 3 in which the groove 21c of the inner layer tube 21 was configured to have a structure in which two spiral grooves intersect each other (hereinafter, referred to as a double spiral groove) was particularly excellent. In Example 3, it is considerable that since the double spiral groove was formed, the number and volume of the grooves were increased such that it becomes easier for bending the inner layer tube 21 and the flexibility was improved as compared with Examples 1 and 2.

Although each preferred embodiment of the present invention has been described above together with each embodiment, the present invention is not limited to this embodiment and each embodiment. Configurations can be added, omitted, replaced, and other modifications without departing from the spirit of the present invention.

Further, the present invention is not limited by the above description and is limited only by the appended claims.

What is claimed is:

1. A tube for a medical device, comprising:
an inner layer tube formed from an elastomer or a resin having flexibility and having a non-parallel groove with respect to an axial direction formed on an outer circumferential surface of the inner layer tube;
an outer layer formed from an elastomer resin softer than a base material of the inner layer tube and configured to fill the groove while covering an outer circumference of the inner layer tube; and
a braid formed from metal wires and only arranged in locations other than the groove in the outer layer,
wherein the outer layer is divided to at least two layers,
the innermost layer is a layer filling the groove of the inner layer tube, and
the braid is arranged only in the layers other than the innermost layer.

2. The tube for a medical device according to claim 1, wherein a material of the inner tube includes polytetrafluoroethylene (PTFE).

3. The tube for a medical device according to claim 1, wherein a material of the outer layer includes fluororubber.

4. A medical device, comprising the tube for a medical device according to claim 1.

5. The medical device according to claim 4, wherein the medical device is an endoscope.
